(19) United States
(12) Patent Application Publication
(10) Pub. No.: US 2024/0373915 A1
(43) Pub. Date: Nov. 14, 2024

(54) AEROSOL-GENERATING DEVICE WITH RETRACTABLE MOUTHPIECE AND STERILIZATION RADIATION SOURCE

(71) Applicant: Philip Morris Products S.A., Neuchatel (CH)

(72) Inventor: Rui Nuno BATISTA, Neuchatel (CH)

(73) Assignee: Philip Morris Products S.A., Neuchatel (CH)

(21) Appl. No.: 18/683,097

(22) PCT Filed: Aug. 18, 2022
(86) PCT No.: PCT/EP2022/073036

§ 371 Date: Feb. 12, 2024

(30) Foreign Application Priority Data
Aug. 25, 2021 (EP) 21193019.3

(51) Int. Cl. A24F 40/40; A24D 1/20; A24F 7/02; A24F 40/20; A24F 40/85; A61L 2/10

(52) U.S. Cl. CPC A24F 40/40; A24D 1/20; A24F 7/02; A24F 40/20; A24F 40/85; A61L 2/10; A61L 2202/11; A61L 2202/16

(57) ABSTRACT

An aerosol-generating device is provided, including: a cavity including an upstream portion with an upstream opening and a downstream portion with a downstream opening; a mouthpiece configured to be retractable into the downstream portion of the cavity; and a sterilization radiation source configured for irradiating at least parts of the downstream portion of the cavity for sterilizing the mouthpiece, the cavity being configured to receive an aerosol-generating article through the upstream opening into the upstream portion, the cavity being further provided with a connection element slidably mounted within the cavity and configured to retract the mouthpiece in the cavity and to push the aerosol-generating article out of the cavity. An aerosol-generating system including the aerosol-generating device and an aerosol-generating article, and a method for operating the aerosol-generating system, are also provided.

The present invention relates to an aerosol-generating device comprising a mouthpiece. The present invention further relates to an aerosol-generating system comprising the aerosol-generating device and an aerosol-generating article. The present invention also relates to a method of operating the aerosol-generating system.

Aerosol-generating devices including mouthpieces are known from the art. After use, user's residual saliva and particles from the aerosol or from the environment may lead to the contamination of the used mouthpiece. This may also result in unpleasant flavors and deterioration of the hygiene associated with the aerosol-generating device.

Aerosol-generating devices comprising a mouthpiece tend to have a large size, making the handling of the devices more difficult for a user.

It would be desirable to provide an aerosol-generating device with a mouthpiece which ensures hygienic conditions of use for the mouthpiece. Furthermore, it would be desirable to provide an aerosol-generating device with a mouthpiece, which is compact. Additionally, it would be desirable to provide an aerosol-generating device with a mouthpiece, wherein after use, the device with the mouthpiece can easily be stored.

According to an embodiment of the present invention there is provided an aerosol-generating device comprising a cavity. The cavity may comprise an upstream portion with an upstream opening. The cavity furthermore may comprise a downstream portion with a downstream opening. The aerosol-generating device may comprise a mouthpiece being configured to be retractable into the downstream portion of the cavity. The aerosol-generating device may comprise a sterilization radiation source configured for irradiating at least parts of the downstream portion of the cavity for sterilizing the mouthpiece. The cavity may be configured for receiving an aerosol-generating article through the upstream opening into the upstream portion.

According to another embodiment of the present invention there is provided an aerosol-generating device comprising a cavity. The cavity comprises an upstream portion with an upstream opening and a downstream portion with a downstream opening. The aerosol-generating device furthermore comprises a mouthpiece being configured to be retractable into the downstream portion of the cavity.

As used herein, the terms “upstream”, and “downstream”, are used to describe the relative positions of components, or portions of components, of the aerosol-generating device with the mouthpiece in relation to the direction in which air flows through the aerosol-generating device and its mouthpiece during use thereof along the air flow path. The aerosol-generating device according to the invention comprise a proximal end through which, in use, an aerosol exits the aerosol-generating device through the mouthpiece. The proximal end of the aerosol generating device may also be referred to as the mouth end or the downstream end. The mouth end is downstream of the distal end. The distal end of the aerosol generating device may also be referred to as the upstream end. Components, or portions of components, of the aerosol generating device may be described as being upstream or downstream of one another based on their relative positions with respect to the airflow path through the aerosol generating device and its mouthpiece.

The aerosol-generating device may allow the insertion and the heating of an aerosol-generating article in the upstream portion of the cavity. Furthermore, the downstream portion of the cavity may serve as a space for receiving the retracted mouthpiece. The sterilization radiation source may sterilize the mouthpiece while being retracted in the downstream portion of the cavity. Such an aerosol-generating device may be very compact. The aerosol-generating device may be hygienic due to the sterilization radiation source. This may allow the sterilization of the mouthpiece and at least parts, or all of the cavity located in the aerosol-generating device.

The mouthpiece may be movably connected with the aerosol-generating device. This may allow the construction of a compact mouthpiece. This may avoid any laborious disassembly and assembly of the aerosol-generating device with the mouthpiece.

The mouthpiece of the aerosol-generating device may be configured arrangeable in a first position in which the mouthpiece is protracted relative to the cavity. The mouthpiece may be configured arrangeable in a second position in which the mouthpiece is retracted in the cavity. This may ease the handling of the mouthpiece in the aerosol-generating device. This may furthermore ease the storing of the mouthpiece in the cavity of the device.

The aerosol-generating device may further comprise a connection element. The connection element may be slidably mounted within the cavity. The mouthpiece may be connected to the connection element. Preferably the mouthpiece may be detachably connectable to the connection element. The mouthpiece may be detachably connectable to the connection element by a plug-in connection.

This may enable an easy slidable mounting of the mouthpiece in the aerosol-generating device. This may also enable a detachable slidable connection between the mouthpiece and the aerosol-generating device.

The cavity of the aerosol-generating device may further be provided with the connection element, wherein the connection element may be slidably mounted within the cavity. The mouthpiece may be connected to the connection element.

The connection element may be configured to be slidably arrangeable in a first position wherein the mouthpiece is protracted relative to the cavity. In the first position the connection element may be located in the downstream portion of the cavity.

This may free the upstream portion of the cavity from the connection element. This may enable the upstream portion of the cavity to receive the aerosol-generating article.

The connection element may be configured to be slidably arrangeable in a second position, wherein the mouthpiece is retracted in the cavity. In the second position the connection element may at least partly be located in the upstream portion of the cavity.

Moving the connection element from the downstream portion, the first position, at least partly to the upstream portion, the second position may enable the downstream portion of the cavity to receive the mouthpiece.

The connection element may have a tubular shape. The connection element may comprise an upstream part, wherein the upstream part is configured for pushing an aerosol-generating article received in the cavity out of the cavity in the second position of the connection element.

This may enable retracting the mouthpiece into the cavity and at the same time pushing an aerosol-generating article out of the cavity.

The aerosol-generating device may furthermore comprise a slider. The slider may be connected to the connection element. Preferably the slider may be configured for being handled by a user. The aerosol-generating device may furthermore comprise a casing. Preferably the slider may be slidably mounted in the casing of the aerosol-generating device.

This may enable a user to slidably move the connection element located in the cavity between the first position and the second position of the connection element.

The casing may partly enclose the cavity. The casing may enclose the upstream part and the downstream part of the cavity. The casing also may enclose the sterilization radiation source. The sterilization radiation source may be located inside the casing adjacent to the cavity. The slider may be located on the outside of the casing. This may ease the handling of the slider by a user.

The casing may comprise an opening. A connection bar may be present in the opening. The connection bar may connect the connection element and the slider. The opening may comprise a slit. The slit may be configured to allow movement of the connection bar and the slider between the first position and the second position of the connection element.

The casing may comprise or be made of polymeric compounds, for example plastics like polyolefines, such as PE, LDPE or PP.

The connection element, the connection bar and the slider may be formed as a one-piece element. This may confer further stability to the slider and the connection element.

The connection element with the connection bar and the slider may be made of polymeric compounds, for example plastic such as PEEK (Polyetheretherketon). Alternatively, the connection element with the slider and the connection bar can be made from glass, or metallic alloy.

The cavity further may comprise walls. At least parts of the walls in the downstream portion of the cavity may be transparent for the radiation emitted by the sterilization radiation source.

This may enable a sterilization of the mouthpiece retracted in the downstream portion of the cavity through the transparent walls of the cavity.

At least parts of the connection element may be made of material being transparent to the radiation of the sterilization radiation source. In particular, the parts of the connection element which are adjacent to the mouthpiece when the mouthpiece is mounted in the connection element may be made transparent.

This may aid in the sterilization of the mouthpiece and may enable the sterilization of parts of the mouthpiece which are configured for being detachably connectable to the connection element.

The mouthpiece may at least partly be transparent for the radiation emitted by the sterilization radiation source. Preferably, the mouthpiece may comprise prismatic elements. The prismatic elements may be configured for propagating the radiation emitted by the sterilization radiation source within the mouthpiece.

This may ease the propagation of the radiation of the sterilization radiation source within the mouthpiece. This may enable large parts or even the complete mouthpiece to be sterilized with ease.

The mouthpiece may comprise or be made of transparent polymeric materials. This may aid in the propagation of the radiation from the sterilization radiation force. The material of the mouthpiece may be polymers, such as polycarbonate (PC).

The mouthpiece may comprise a Venturi element. The Venturi element may be located at the upstream end of the mouthpiece.

The Venturi element may enable a cooling of the aerosol formed from the aerosol-forming substrate of the aerosol-generating device. The aerosol may be cooled before being consumed by the user.

The Venturi element may comprise an airflow channel. The airflow channel may comprise an inlet portion, a central portion and an outlet portion. The inlet portion may be configured converging towards the central portion. The outlet portion may be configured diverging from the central portion. The inlet portion may be located upstream of the outlet portion.

This may enable an efficient cooling of the aerosol. This may enable an efficient mixing of air with the aerosol formed from the aerosol-generating article.

An extension of the outlet portion within the mouthpiece may be larger than an extension of the inlet portion within the mouthpiece. This may enable an efficient mixing and cooling of the aerosol.

A cross-sectional area at the upstream end of the mouthpiece may be smaller than a cross-sectional area at the downstream end of the mouthpiece. This may enable an efficient mixing and cooling of the aerosol.

The aerosol-generating device may comprise an airflow path. The airflow path may lead from the upstream opening of the cavity through the connection element to the mouthpiece. The airflow path may furthermore lead from the inlet portion of the mouthpiece through the central portion to the outlet portion of the mouthpiece.

This enables an efficient airflow path through the aerosol-generating device and its mouthpiece.

The aerosol-generating device may further comprise a downstream cover. The downstream cover may be configured to cover the downstream opening of the cavity, when the mouthpiece is retracted in the cavity. The downstream cover may comprise a material being opaque for the radiation emitted by the sterilization radiation source.

The downstream cover may avoid contamination of the cavity and of the mouthpiece retracted in the downstream part of the cavity. The downstream cover may avoid the propagation of the radiation emitted by the sterilization radiation source outside the cavity and the mouthpiece during the sterilization procedure.

The material of the downstream cover may comprise plastic, metal or ceramic.

The downstream cover may be configured slidable over a downstream end face of the aerosol-generating article. The downstream end face may include the downstream opening.

The aerosol-generating device may further comprise an upstream cover. The upstream cover may be configured to close the upstream opening of the upstream portion of the cavity. This may avoid any contamination of the upstream portion of the cavity.

The upstream cover may be configured to close the upstream opening of the upstream portion of the cavity when an aerosol-generating article is received in the upstream portion of the cavity. The aerosol-generating article therefore may have a length corresponding or being smaller than the length of the upstream portion of the cavity.

The upstream cover may be configured to either be slidable or be rotatable over the upstream end face of the aerosol-generating article.

The upstream cover may be made of polymers such as PCTFE (polychloro-trifluoroethylene).

The aerosol-generating device may further comprise a control unit. The control unit may be configured for controlling the sterilization radiation source. The control unit may be configured for activating the sterilization radiation source when the mouthpiece is retracted in the cavity.

This may enable an efficient controlling of the sterilization radiation source. This may ensure that the sterilization radiation source is not accidentally activated when the aerosol-generating device is used by a user for inhaling the aerosol.

The control unit may be configured for activating the sterilization radiation source for a specified time range. The specified time range may be from 0.5 seconds to 1 minute, preferably from 11 seconds to 17 seconds, most preferably from 11 seconds to 13 seconds.

Such a time range may enable an efficient sterilization of the mouthpiece. Such a time range may also enable an efficient sterilization of at least parts of the cavity. This time range furthermore may avoid that the sterilization radiation source is activated for a prolonged time when the mouthpiece is retracted in the downstream portion of the cavity. This may save energy. Furthermore, this may avoid the material of the casing of the aerosol-generating device and—if present—the material of the downstream cover to be exposed to the radiation of the sterilization radiation source for a prolonged time. This may avoid or reduce any degradation of one or both of the material of the casing of the aerosol-generating device and the material of the downstream cover due to the radiation of the sterilization radiation source.

The control unit also may be configured to control the heating element, as described below in greater detail.

The sterilization radiation source may be configured for emitting UV radiation. Preferably the UV radiation may be in the range from 100 to 280 nanometers. This may enable an efficient sterilization of the retracted mouthpiece. This also may enable an efficient sterilization of the cavity.

The sterilization radiation source may comprise an array of one or both of UV radiation emitting light emitting diodes (LEDs) and UV radiation emitting organic light emitting diodes (OLEDs). These arrays may enable to subject large parts of the mouthpiece and the cavity to the radiation emitted by the sterilization radiation source.

The aerosol-generating device may further comprise a heating element. The heating element may be located at least partly around the cavity for heating an aerosol-generating article received in the cavity. The heating element may be configured for heating the aerosol-generating article below a combustion temperature of an aerosol-forming substrate.

The heating element may be configured to heat an aerosol-generating article received in the cavity. The heating element may be configured to heat the aerosol-generating article to a temperature ranging from 220 degrees Celsius to 400 degrees Celsius, preferably from 250 degrees Celsius to 290 degrees Celsius. At these temperatures an aerosol may be generated from the aerosol-forming substrate included in the aerosol-generating article.

The heating element may take any suitable form. For example, a heating element may take the form of one or more flexible heating foils on a dielectric substrate, such as polyimide. The flexible heating foils can be shaped to conform to the perimeter of the cavity. Alternatively, an external heating element may take the form of a metallic grid or grids, a flexible printed circuit board, a molded interconnect device (MID), ceramic heater, flexible carbon fibre heater or may be formed using a coating technique, such as plasma vapour deposition, on a suitable shaped substrate. A heating element may also be formed using a metal having a defined relationship between temperature and resistivity. In such an exemplary device, the metal may be formed as a track between two layers of suitable insulating materials. A heating element formed in this manner may be used to both heat and monitor the temperature of the heating element during operation. The heating element may be configured as a resistive heating element. The heating element may be configured as a resistive heating coil, at least partly surrounding the cavity, in particular the upstream part of the cavity receiving the aerosol-generating article.

The sterilization radiation source of the aerosol-generating device may be arranged adjacent to the downstream portion of the cavity. The sterilization radiation source may at least partly surround the downstream portion of the cavity.

This may ensure an efficient sterilization of the mouthpiece received in the downstream portion of the cavity.

The upstream portion and the downstream portion of the cavity may be fluidly connected. The aerosol-generating device may comprise a common central longitudinal axis running through the aerosol-generating device. The upstream portion and the downstream portion of the cavity may be arranged along the common central longitudinal axis. Preferably, the upstream portion and the downstream portion may form one continuous central cavity of the aerosol-generating device.

This may ease operating the aerosol-generating device. This may enable an easy and efficient cleaning of the one continuous central cavity of the aerosol-generating device. This may enable a direct flow path between the upstream portion of the cavity which is configured for receiving the aerosol-generating article and the downstream portion of the cavity with the mouthpiece.

The aerosol-generating device may comprise a power supply, typically a battery, within the casing of the aerosol-generating device. In one embodiment, the power supply is a Lithium-ion battery. Alternatively, the power supply may be a Nickel-metal hydride battery, a Nickel cadmium battery, or a Lithium based battery, for example a Lithium-Cobalt, a Lithium-Iron-Phosphate, Lithium Titanate or a Lithium-Polymer battery. As an alternative, the power supply may be another form of charge storage device such as a capacitor. The power supply may require recharging and may have a capacity that enables to store enough energy for one or more usage experiences; for example, the power supply may have sufficient capacity to continuously generate aerosol for a period of around six minutes or for a period of a multiple of six minutes. In another example, the power supply may have sufficient capacity to provide a predetermined number of puffs or discrete activations of the heating element.

The aerosol-generating device may comprise electric circuitry. The electric circuitry may comprise a microprocessor, which may be a programmable microprocessor. The microprocessor may be part of a control unit. The electric circuitry may comprise further electronic components. The electric circuitry may be configured to regulate a supply of power to the heating element, particularly to the resistive heating coil. Power may be supplied to the heating element continuously following activation of the aerosol-generating device or may be supplied intermittently, such as on a puff-by-puff basis. The power may be supplied to the heating element in the form of pulses of electrical current. The electric circuitry may be configured to monitor the electrical resistance of the heating element, and preferably to control the supply of power to the heating element dependent on the electrical resistance of the heating element.

The control unit also may be configured to control the activation of the sterilization radiation source. The control unit may be configured to activate the sterilization radiation source while the aerosol-generating device is operated by a user. This may avoid any harm to the user from the radiation of the sterilization radiation source. The control unit may be configured to activate the sterilization radiation source upon one or both of: retracting the mouthpiece into the cavity and closing the downstream cover.

Another embodiment provides an aerosol-generating system. The aerosol-generating system may comprise an aerosol-generating device as described herein. The aerosol-generating system may additionally comprise an aerosol-generating article.

A further embodiment provides an aerosol-generating system. The aerosol-generating system comprises an aerosol-generating device as described herein. Furthermore, the aerosol-generating system comprises an aerosol-generating article.

Such an aerosol-generating system may enable a user to easily operate an aerosol-generating device with a mouthpiece. This may also enable a user to store the aerosol-generating device in a compact form.

In the aerosol-generating system the upstream part of the connection element may be configured to be located adjacent to the aerosol-generating article received in the cavity.

This may enable a user to easily eject a used aerosol-generating article from the cavity by operating the connection element and pushing the connection element from its first position to its second position.

The aerosol-generating article may comprise a substrate section including aerosol-forming substrate. The aerosol-forming substrate may comprise solid material.

As used herein, the term ‘aerosol-forming substrate’ relates to a substrate capable of releasing one or more volatile compounds that can form an aerosol. Such volatile compounds may be released by heating the aerosol-forming substrate.

The aerosol-forming substrate may comprise liquid components. The aerosol-forming substrate may comprise a tobacco-containing material containing volatile tobacco flavour compounds which are released from the substrate upon heating. The aerosol-forming substrate may comprise tobacco extract. The aerosol-forming substrate may comprise a non-tobacco material. The aerosol-forming substrate may comprise an aerosol former that facilitates the formation of a dense and stable aerosol. Suitable aerosol-formers are well known in the art and include, but are not limited to: polyhydric alcohols, such as triethylene glycol, 1,3-butanediol and glycerine; esters of polyhydric alcohols, such as glycerol mono-, di- or triacetate; and aliphatic esters of mono-, di- or polycarboxylic acids, such as dimethyl dodecanedioate and dimethyl tetradecanedioate. Aerosol formers may be polyhydric alcohols or mixtures thereof, such as triethylene glycol, 1,3-butanediol and glycerine. The aerosol-former may be propylene glycol. The aerosol former may comprise both glycerine and propylene glycol.

Another embodiment provides a method for operating an aerosol-generating system as described herein. The method may comprise drawing the mouthpiece through the downstream opening out of the downstream portion of the cavity. The method furthermore may comprise inserting an aerosol-generating article into the upstream portion of the cavity through the upstream opening. The method may furthermore comprise consuming the aerosol-generating article.

A further embodiment provides a method for operating an aerosol-generating system as described herein. The method comprises the step of drawing the mouthpiece through the downstream opening out of the downstream portion of the cavity. The method furthermore comprises the method step of inserting the aerosol-generating article into the upstream portion of the cavity through the upstream opening. The method furthermore comprises consuming the aerosol-generating article.

The method furthermore may comprise an additional method step of retracting the mouthpiece into the cavity after consumption of the aerosol-generating article. This may activate the sterilization radiation source for sterilizing the retracted mouthpiece.

In the method of operating the aerosol-generating system retracting the mouthpiece into the cavity may cause the upstream part of the connection element to slidably move into the downstream portion of the cavity. This may push the aerosol-generating article out of the cavity.

Such a method may ease the automatic ejection of a used aerosol-generating article from the cavity. A user may not be required to manually remove the aerosol-generating article from the cavity.

Below, there is provided a non-exhaustive list of non-limiting examples. Any one or more of the features of these examples may be combined with any one or more features of another example, embodiment, or aspect described herein.

Example A: Aerosol-generating device, comprising
a cavity comprising an upstream portion with an upstream opening and a downstream portion with a downstream opening,
a mouthpiece being configured to be retractable into the downstream portion of the cavity, and
a sterilization radiation source configured for irradiating at least parts of the downstream portion of the cavity for sterilizing the mouthpiece,
the cavity being configured for receiving an aerosol-generating article through the upstream opening into the upstream portion.

Example B: Aerosol-generating device according to the preceding example, wherein the mouthpiece is movably connected with the aerosol-generating device, preferably wherein the mouthpiece is configured arrangeable in a first position in which the mouthpiece is protracted relative to the cavity, and
wherein the mouthpiece is configured arrangeable in a second position in which the mouthpiece is retracted in the cavity.

Example C: Aerosol-generating device according to the preceding example, further comprising a connection element, the connection element being slidably mounted within the cavity, wherein the mouthpiece is connected to the connection element, preferably wherein the mouthpiece is detachably connected to the connection element.

Example D: Aerosol-generating device according to the preceding example, wherein the connection element is configured to be slidably arrangeable in a first position wherein the mouthpiece is protracted relative to the cavity, wherein in the first position the connection element is located in the downstream portion of the cavity and
wherein the connection element is configured to be slidably arrangeable in a second position, wherein the mouthpiece is retracted in the cavity wherein in the second position of the connection element is at least partly located in the upstream portion of the cavity.

Example E: Aerosol-generating device according to the preceding example, wherein the connection element has a tubular shape, the connection element comprising an upstream part, wherein the upstream part is configured for pushing an aerosol-generating article received in the cavity out of the cavity in the second position.

Example F: Aerosol-generating device according to any of the preceding Examples C to E, further comprising a slider, the slider being connected to the connection element, preferably wherein the slider is configured for being handled by a user, more preferably wherein the slider is slidably mounted in a casing of the aerosol-generating device.

Example G: Aerosol-generating device according to any of the preceding examples, the cavity further comprises walls, wherein at least parts of the walls in the downstream portion of the cavity are transparent for the radiation emitted by the sterilization radiation source.

Example H: Aerosol-generating device according to any of the preceding examples, wherein the mouthpiece is at least partly transparent for the radiation emitted by the sterilization radiation source, preferably wherein the mouthpiece comprises prismatic elements configured for propagating the radiation emitted by the sterilization radiation source within the mouthpiece.

Example I: Aerosol-generating device according to any of the preceding examples, wherein the mouthpiece comprises a Venturi element, preferably wherein the Venturi element is located at the upstream end of the mouthpiece.

Example J: Aerosol-generating device according to any of the preceding examples, wherein a cross-sectional area at the upstream end of the mouthpiece is smaller than a cross-sectional area at the downstream end of the mouthpiece.

Example K: Aerosol-generating device according to any of the preceding examples, comprising an airflow path leading from the upstream opening through the connection element to the mouthpiece.

Example L: Aerosol-generating device according to any of the preceding examples, further comprising a downstream cover, the downstream cover being configured to cover the downstream opening of the cavity, when the mouthpiece is retracted in the cavity, preferably wherein the downstream cover comprises a material being opaque for the radiation emitted by the sterilization radiation source.

Example M: Aerosol-generating device according to any of the preceding examples, further comprising a control unit configured for controlling the sterilization radiation source, preferably wherein the control unit is configured for activating the sterilization radiation source when the mouthpiece is retracted in the cavity.

Example N: Aerosol-generating device according to any of the preceding examples, wherein the sterilization radiation source is configured for emitting UV radiation, preferably UV radiation in the range from 100 to 280 nm.

Example O: Aerosol-generating device according to any of the preceding examples, wherein the sterilization radiation source comprises an array of one or both of: UV radiation emitting LEDs and UV radiation emitting OLEDs.

Example P: Aerosol-generating device according to any of the preceding examples, further comprising a heating element located at least partly around the cavity for heating an aerosol-generating article received in the cavity, preferably wherein the heating element is configured for heating the aerosol-generating article below a combustion temperature of an aerosol-forming substrate.

Example Q: Aerosol-generating device according to any of the preceding examples, wherein the sterilization radiation source is arranged adjacent to the downstream portion of the cavity, preferably wherein the sterilization radiation source at least partly surrounds the downstream portion of the cavity.

Example R: Aerosol-generating device according to any of the preceding examples, wherein the upstream portion and the downstream portion of the cavity are fluidly connected, preferably wherein the upstream portion and the downstream portion are arranged along a common central longitudinal axis of the aerosol-generating device, more preferably wherein the upstream portion and the downstream portion form one continuous central cavity of the aerosol-generating device.

Example S: Aerosol-generating system, comprising
an aerosol-generating device according to any of the preceding examples, and an aerosol-generating article.

Example T: Aerosol-generating system according to the preceding claim further according to claim E, wherein the upstream part of the connection element is configured to be located adjacent to the aerosol-generating article received in the cavity.

Example U: Aerosol-generating system according to any of the preceding examples S or T, wherein the aerosol-generating article comprises a substrate section including aerosol-forming substrate, preferably wherein the aerosol-forming substrate comprises solid material.

Example V: Method for operating an aerosol-generating system according to any of the preceding examples S to U comprising,
drawing the mouthpiece through the downstream opening out of the downstream portion of the cavity,
inserting the aerosol-generating article into the upstream portion of the cavity through the upstream opening, and
consuming the aerosol-generating article.

Example W: Method for operating an aerosol-generating system according to the preceding example, further comprising
retracting the mouthpiece into the cavity after consumption of the aerosol-generating article, and
activating the sterilization radiation source for sterilizing the retracted mouthpiece.

Example X: Method for operating an aerosol-generating system according to the preceding example, wherein retracting the mouthpiece into the cavity causes the upstream part of the connection element to slidably move into the upstream portion of the cavity, thereby pushing the aerosol-generating article out of the cavity.

Features described in relation to one embodiment may equally be applied to other embodiments of the invention.

The invention will be further described, by way of example only, with reference to the accompanying drawings in which:

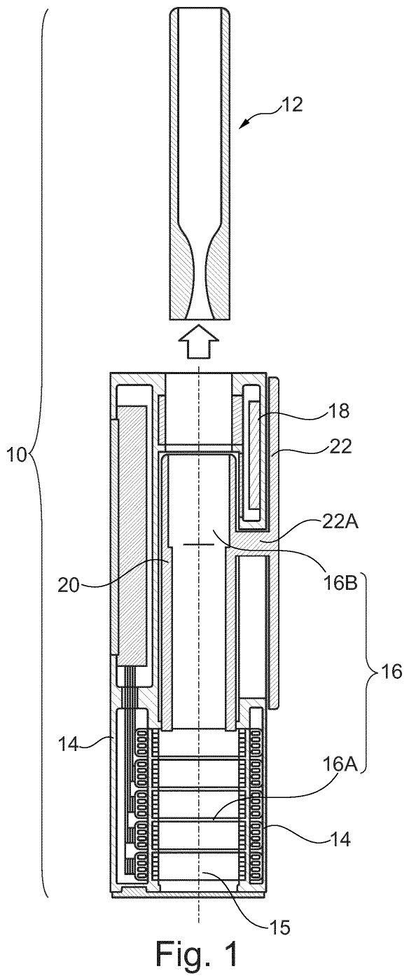

FIG. 1 shows a cross-sectional view of an aerosol-generating device including the mouthpiece;

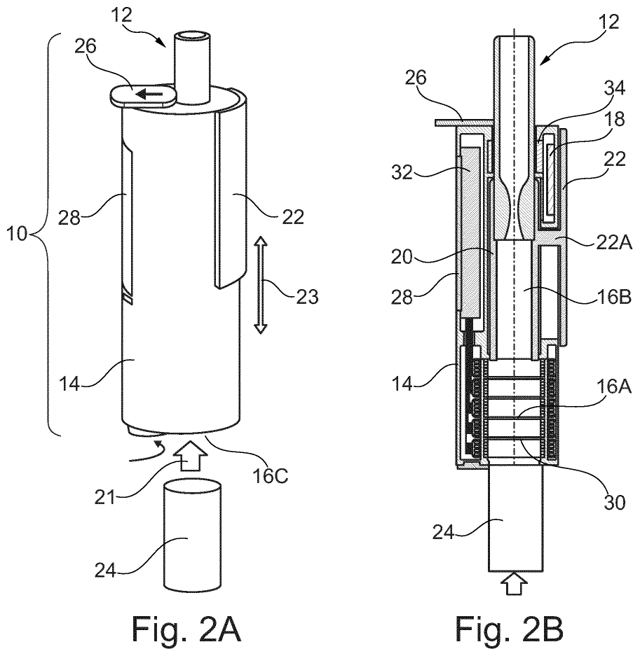

FIGS. 2A and 2B depict a perspective view of an aerosol-generating device with a protracted mouthpiece and a cross-sectional view of the same aerosol-generating device, respectively;

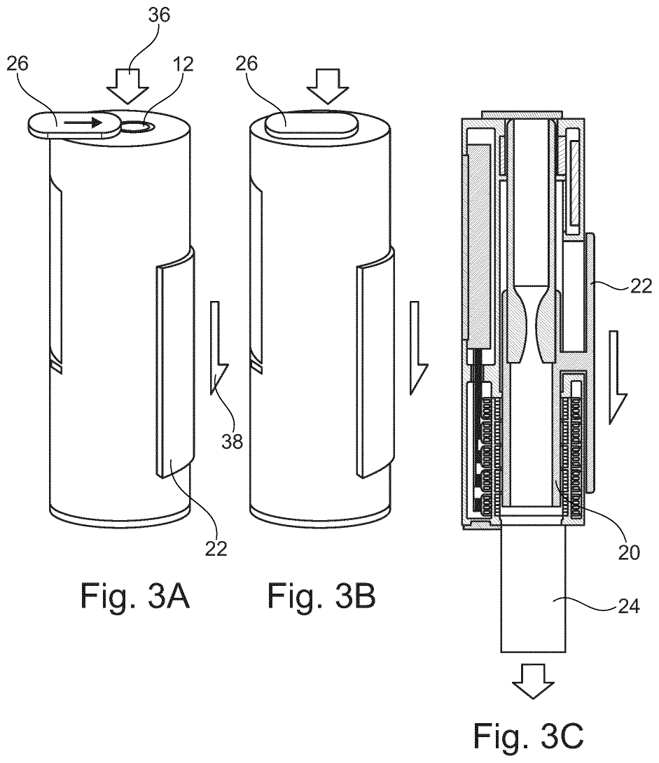

FIG. 3A to 3C show a method of retracting the mouthpiece into the cavity and closing the downstream cover and ejecting the aerosol-generating article from the cavity of the aerosol-generating device.

In the following the same elements are marked with the same reference numerals throughout all the figures.

FIG. 1 depicts a schematic cross-sectional view of one embodiment of an aerosol-generating device 10 in accordance with the present invention, wherein its mouthpiece 12 has been detached from connection element 20 inside the cavity 16. The casing 14 of the aerosol-generating device includes a cavity 16 with an upstream portion 16A of the cavity and with a downstream portion 16B of the cavity. The upstream portion 16A and the downstream portion 16B of the cavity form one continuous cavity along the common central longitudinal axis 15 of the aerosol-generating device indicated by the dashed line. The downstream portion 16B of the cavity accommodates the connection element 20 to which the mouthpiece 12 can be detachably connected. The connection element 20 is connected to a slider 22 via the connection bar 22A. The slider 22 can be manually moved in order to move the connection element 20 inside the cavity 16 of the aerosol-generating device. A sterilization radiation source 18 is present inside the casing 14, which is configured to irradiate the mouthpiece 12, when it is retracted in the downstream portion 16B of the cavity. The arrow indicates the direction of detaching the mouthpiece 12 from the connection element 20.

FIG. 2A shows a perspective view of an aerosol-generating system, additionally including the aerosol-generating article 24. The mouthpiece 12 is protracted relative to the cavity and to the casing 14 and can be used by a user for inhaling an aerosol generated from the aerosol-generating article. A movable downstream cover 26 is opened in order to allow the protraction of the mouthpiece 12. The slider 22 is in the upper position, so that the connection element 20 is located in the downstream part of the cavity (cavity and connection element not shown in FIG. 2A). The double arrow 23 indicates the two directions of movement for the slider. The aerosol-generating article 24 can be inserted into the upstream part of the cavity through the upstream opening 16C of the cavity as indicated by the arrow 21. The aerosol-generating device additionally includes a display 28.

FIG. 2B depicts a cross-sectional view of the aerosol-generating system after the aerosol-generating article 24 has been inserted into the upstream part of the cavity. The upstream part of the cavity contains a heating element in the form of a heater coil 30. The heater coil 30 is configured for heating the substrate portion of the aerosol-generating article containing the aerosol-forming substrate to a temperature below the combustion temperature of the substrate. This generates an aerosol to be inhaled by a user. The aerosol-generating device includes the sterilization radiation source 18, which is configured to irradiate the mouthpiece 12 through the transparent window 34 in the inner wall of the cavity, when the mouthpiece is retracted in the cavity. The aerosol-generating device additionally includes a power supply 32. The aerosol-generating system of FIG. 2B is ready for operation and allows a user to inhale an aerosol by sucking on the mouthpiece 12

FIG. 3A depicts a perspective view of an aerosol-generating device after the mouthpiece 12 has been retracted into the downstream part of the cavity indicated by the arrow 36. The downstream cover 26 is still open, so that the retracted mouthpiece 12 is visible. The mouthpiece has been retracted into the cavity by moving the slider 22 into upstream direction, as indicated by the arrow 38.

FIG. 3B shows the device of FIG. 3A, wherein the downstream cover 26 has been closed.

FIG. 3C shows a schematic cross-sectional view of the aerosol-generating system, wherein the aerosol-generating article 24 has been pushed out of the cavity of the aerosol-generating device by moving the slider 22 in upstream direction as shown in FIG. 3A. Moving the slider 22 in upstream direction therefore can retract the mouthpiece 12 into the downstream portion of the cavity of the aerosol-generating device and can at the same time push the aerosol-generating article out of the upstream portion of the cavity.

1.-15. (canceled)

16. An aerosol-generating device, comprising:
a cavity comprising an upstream portion with an upstream opening and a downstream portion with a downstream opening;
a mouthpiece configured to be retractable into the downstream portion of the cavity; and
a sterilization radiation source configured for irradiating at least parts of the downstream portion of the cavity for sterilizing the mouthpiece,
wherein the cavity is configured to receive an aerosol-generating article through the upstream opening into the upstream portion,
wherein the cavity is further provided with a connection element being slidably mounted within the cavity and configured to retract the mouthpiece in the cavity and to push the aerosol-generating article out of the cavity.

17. The aerosol-generating device according to claim 16, wherein the mouthpiece is movably connected with the aerosol-generating device.

18. The aerosol-generating device according to claim 17,
wherein the mouthpiece is further configured to be arrangeable in a first position in which the mouthpiece is protracted relative to the cavity, and
wherein the mouthpiece is further configured to be arrangeable in a second position in which the mouthpiece is retracted in the cavity.

19. The aerosol-generating device according to claim 17, wherein the mouthpiece is detachably connected to the connection element.

20. The aerosol-generating device according to claim 16,
wherein the connection element is configured to be slidably arrangeable in a first position in which the mouthpiece is protracted relative to the cavity,
wherein in the first position the connection element is located in the downstream portion of the cavity,
wherein the connection element is configured to be slidably arrangeable in a second position in which the mouthpiece is retracted in the cavity, and
wherein in the second position of the connection element is at least partly located in the upstream portion of the cavity.

21. The aerosol-generating device according to claim 20,
wherein the connection element has a tubular shape and comprises an upstream part, and
wherein the upstream part is configured for pushing the aerosol-generating article received in the cavity out of the cavity in the second position.

22. The aerosol-generating device according to claim 16, wherein the mouthpiece comprises a Venturi element.

23. The aerosol-generating device according to claim 22, wherein the Venturi element is located at an upstream end of the mouthpiece.

24. The aerosol-generating device according to claim 16, further comprising a downstream cover, the downstream cover being configured to cover the downstream opening of the cavity, when the mouthpiece is retracted in the cavity.

25. The aerosol-generating device according to claim 24, wherein the downstream cover comprises a material being opaque to radiation emitted by the sterilization radiation source.

26. The aerosol-generating device according to claim 16, wherein the sterilization radiation source is configured to emit UV radiation.

27. The aerosol-generating device according to claim 26, wherein the sterilization radiation source is configured to emit UV radiation in a wavelength range from 100 nm to 280 nm.

28. The aerosol-generating device according to claim 16, wherein the sterilization radiation source comprises an array of one or both of: UV radiation emitting LEDs and UV radiation emitting OLEDs.

29. An aerosol-generating system, comprising an aerosol-generating device according to claim 16 and an aerosol-generating article.

30. The aerosol-generating system according to claim 29,
wherein the connection element of the aerosol-generating device has a tubular shape,
wherein the connection element of the aerosol-generating device comprises an upstream part,
wherein the upstream part is configured for pushing the aerosol-generating article received in the cavity out of the cavity in the second position, and
wherein the upstream part is further configured to be located adjacent to the aerosol-generating article received in the cavity.

31. The aerosol-generating system according to claim 29, wherein the aerosol-generating article comprises a substrate section including aerosol-forming substrate.

32. The aerosol-generating system according to claim 31, wherein the aerosol-forming substrate comprises solid material.

33. A method for operating an aerosol-generating system according to claim 29, the method comprising:
drawing the mouthpiece of the aerosol-generating device through the downstream opening out of the downstream portion of the cavity;
inserting the aerosol-generating article into the upstream portion of the cavity through the upstream opening; and
consuming the aerosol-generating article.

34. The method for operating an aerosol-generating system according to claim 33, further comprising
retracting the mouthpiece into the cavity after consumption of the aerosol-generating article; and
activating the sterilization radiation source for sterilizing the retracted mouthpiece.

35. The method for operating an aerosol-generating system according to claim 34, wherein the retracting of the mouthpiece into the cavity causes the upstream part of the connection element to slidably move into the upstream portion of the cavity, thereby pushing the aerosol-generating article out of the cavity.
